(19) United States
(12) Patent Application Publication
(10) Pub. No.: US 2026/0196294 A1
(43) Pub. Date: Jul. 9, 2026

(54) Computer-Implemented Means and Methods for the Prediction of the Stability of Proteins and Protein Complexes

(71) Applicants: VIB VZW, Gent (BE); Katholieke Universiteit Leuven, Leuven (BE); Fundació Centre De Regulació Genòmica, Barcelona (ES); Institució Catalana De Recerca I Estudis Avançats, Barcelona (ES)

(72) Inventors: Frederic Rousseau, Leuven (BE); Joost Schymkowitz, Leuven (BE); Gabriele Orlando, Leuven (BE); Luis Serrano Pubul, Barcelona (ES)

(21) Appl. No.: 19/132,958

(22) PCT Filed: Nov. 28, 2023
(86) PCT No.: PCT/EP2023/083289

§ 371 Date: May 27, 2025

(30) Foreign Application Priority Data
Nov. 28, 2022 (EP) 22209958.2

(51) Int. Cl. G16B 15/20; G16B 40/00

(52) U.S. Cl. CPC G16B 15/20; G16B 40/00

(57) ABSTRACT

The present methods and systems generally relate to the biomedical field and relate to subfields of computational biology and bioinformatics. More, specifically the invention provides an artificial intelligence algorithm which can predict the stability of proteins and protein complexes.

### CROSS REFERENCE TO RELATED APPLICATIONS

This application is a national phase entry under 35 U.S.C. § 371 of International Patent Application PCT/EP2023/083289, filed Nov. 28, 2023, designating the United States of America and published in English as International Patent Publication WO2024/115452 on Jun. 6, 2024, which claims the benefit under Article 8 of the Patent Cooperation Treaty to European Patent Application Serial No. 22209958.2, filed Nov. 28, 2022, the entireties of which are hereby incorporated by reference.

### FIELD OF THE INVENTION

The present methods and systems generally relate to the biomedical field and relate to subfields of computational biology and bioinformatics. More, specifically the invention provides a computer-implemented algorithm which can predict the stability of proteins and protein complexes and can be integrated in artificial intelligence tools in an end-to-end fashon. Introduction to the invention The protein structure prediction algorithm AlphaFold [1] started a revolution in computational biology by allowing researchers to rapidly access reliable structural information for virtually any natural protein. The general protein folding problem, however, is still far from being solved: while we can predict the conformations of well studied proteins, inferring the structure of orphan or artificial proteins is still technically impossible [2]. This problem also occurs in cases in which the prediction of a protein structure is not the major prediction task, but it is intrinsically connected with it. This is the case, for instance, for docking, in which the receptor-ligand complex is intrinsically connected with affinity, which is the major prediction task. In all these tasks, machine learning (ML) algorithms struggle to converge to meaningful solutions because of the general scarcity of experimental data, id est the algorithms must learn complex physical rules from a limited number of examples, which often results in overfitting. Currently, the only currently available solution is to significantly limit the number of trainable parameters in the ML model, thereby reducing the risk of overfitting but also limiting the learning ability of the algorithm.

In the present invention we have developed an alternative solution to this problem. First, we have defined a set of biophysical rules and encoded them so that they could to be integrated directly in deep learning (DL) algorithms, therefore freeing them from the daunting task of learning physical rules from a few examples. Usually such sets of biophysical rules are defined as ‘force fields’ and they are included in tools such as Rosetta [3] and FoldX [4]. The problem is that traditional force fields are not suitable to interact with DL algorithms due to their implementation: DL algorithms require the calculation of gradients with respect to the neural network (NN) weights in order to perform the learning process. This is only possible when the force field is implemented with data-structures that record the gradient of every operation performed from the input to the output of the DL pipeline. The simple inclusion of a traditional force field inside a DL algorithm would not work and would therefore block the gradient propagation, arresting the training as well. However, in the present invention we have found a solution for this problem which is differentiability and we refer this “differentiability step” to the capability of recording the gradient and transferring it to the next step of a DL pipeline. In addition, DL training is often very computational intensive and a force field needs to be as efficient as possible in both energy and gradient calculations in order to be used in DL settings. This can be achieved by implementing every physical calculation as operations between tensors, for which functions, highly optimized at the hardware level, are available (i.e. CUDA). In the present invention we have developed a computer-implemented method which fulfills all these requirements and this method is herein further designated as MadraX. Specifically here, MadraX is a knowledge-based force field implemented as a PyTorch [5] module, a very popular DL library. The calculation of biophysical rules is inspired by FoldX [4], but unlike FoldX our system is completely differentiable via the autograd function of PyTorch and it can thus be embedded within any PyTorch neural network, in the same way it would be done with a standard NN layer but maintaining all the properties typical of Pytorch data structures. MadraX is useful for example for predicting the folding of a protein or predicting the folding of a protein complex. Other uses of MadraX are depicted in FIGS. 1A and 1B and are further described in the appended examples section.

### DESCRIPTION OF THE FIGURES

The patent or application file contains at least one drawing executed in color. Copies of this patent or patent application publication with color drawing(s) will be provided by the Office upon request and payment of the necessary fee.

The drawings described are only schematic and are non-limiting. In the drawings, the size of some of the elements may be exaggerated and not drawn on scale for illustrative purposes.

FIGS. 1A and 1B: Typical usages of MadraX. MadraX takes a protein structure as input and estimates the energies and atomic interactions that stabilize the fold. It provides a differentiable, tensor-based per-residue energetic landscape of the protein that can directly be used for ML applications. Since MadraX is fully differentiable, its conformational energy estimation can be used to optimize protein structures, applying rotations to the residue torsion angles to find the optimal conformation. MadraX is implemented as a PyTorch module and can therefore be inserted in any NN as easily as any other standard PyTorch NN layer, with the only difference that MadraX has no learned parameters. MadraX can be used together with other DL supporting tools, such as PyUUL [6] and it can provide physical constraints to the network learning process, for example in the refinements of cryo-EM models.

FIG. 2: Performances of protein stability prediction task. Left, correlation of the predicted and experimental ΔΔG. The Pearson's correlation coefficient is 0.51. The x represents the 10% of the validation data that deviates the most from this correlation. Right, box plots of the scores assigned to mutations that destabilize and stabilize the protein folding.

FIG. 3: Performances of the prediction of residues in the binding pocket. Left, ROC curve of the presented method on the test dataset. The area under the curve (AUC) is 0.92. Right, box plots of the scores assigned to residues inside and outside the binding pocket. The orange points show 1000 randomly sampled residues in the two groups and show the actual distribution of the scores.

FIG. 4: RMSF of the simulation of the human SH3 domain. The red bars represent the beta strand segments. Panel A shows the RMSF of the unaligned intermediate conformations, while Panel B shows the same results after aligning the intermediate conformations. The figures should be compared with FIG. 3 of [21].

### DETAILED DESCRIPTION OF THE INVENTION

Deep learning algorithms applied to structural biology often struggle to converge to meaningful solutions when limited data is available, since they are required to learn complex physical rules from examples. And state-of-the-art force-fields cannot interface with deep learning algorithms due to their implementation. In the instant invention we present a computer-implemented tool which was have designated as MadraX, which tool is essentially a forcefield implemented as a differentiable PyTorch module, which is able to interact with deep learning algorithms in an end-to-end fashion.

Thus the present disclosure relates generally to a machine learning engine, herein further referred to as the MadraX algorithm (or in short MadraX), for predicting the stability of the folding of a protein or a protein complex. The present disclosure also relates to a system (or apparatus) implementing the artificial intelligence (AI) platform.

Example embodiments will be described more fully hereinafter, in which example embodiments are described. It should be understood that such systems, computer readable media, and methods may be embodied in many different forms and should not be construed as limited to the example embodiments set forth herein. Rather, these example embodiments are provided so that this disclosure will be thorough and complete, and will fully convey the scope of the claims to those of ordinary skill in the art.

The term “machine learning” as used herein generally refers to a type of artificial intelligence (AI) that provides computers with the ability to learn without being explicitly programmed. Machine learning is a branch of AI focusing on systems that can learn from data, identify patterns, and make decisions with minimal human intervention.

As used herein, the term “full length native protein” refers to a protein that is in its native or natural state and unaltered by any denaturing agent such as heat, chemical mutation or enzymatic reactions. A wild-type protein would be considered a full-length native protein. The term full-length native protein sequence, as used herein, refers to the amino acid sequence found in the full-length native protein.

As used herein “mutation” refers to a change in the amino acid sequence of a native protein. Mutations can be described by using the native sequence and then identifying the specific acid that have been changed. A “mutant” refers to the protein that contains the mutation. A full-length mutant sequence refers to the full amino acid sequence of the mutant protein, instead of describing the mutant as the amino acids that are different from the native protein.

Terms such as “first”, “second”, and “within” are used merely to distinguish one component (or part of a component or state of a component) from another. Such terms are not meant to denote a preference or a particular orientation and are not meant to limit embodiments of the disclosure. In the following detailed description of the example embodiments, numerous specific details are set forth in order to provide a more thorough understanding of the invention. However, it will be apparent to one of ordinary skill in the art that embodiments of the disclosure may be practiced without these specific details. In other instances, well-known features have not been described in detail to avoid unnecessarily complicating the description.

A user may be any person or entity that interacts with the database, the AI platform, or both. Examples of a user may include, but are not limited to, a principal investigator, a scientist, a graduate student, or a pharmaceutical company, for example. There can be one or multiple users.

The present invention provides in a first embodiment a computer-implemented method for predicting the stability of the folding of a protein or a protein complex, said method comprising:
obtaining the crystallographic atom coordinates of a protein structure or the crystallographic coordinates of the structure of a protein complex,
calculating biophysical energy values of each of the amino acid residue interactions present in said protein or protein complex wherein said parameters comprise calculation of hydrogen bonds, electrostatic interactions, polar solvation, hydrophobic solvation, atomic clashes, disulfide bonds, backbone entropy, side chain entropy and other Van der Waals interactions with water, and encoding said energy parameters in higher dimensional tensor data structures, and
introducing said tensor data structures into a deep learning algorithm in an end-to-end manner,
calculating the gradient of said biophysical energy values with respect to a variation in the atom coordinates obtained in step a) or another arbitrary variable with the use of an automatic differentiation engine,
predicting the stability of the folding of a protein or a protein complex.r

The wording “a variation in the atom coordinates” is generally an in silico induced variation or an experimentally induced variation (such as a variation due to a mutation) or a variation due to the binding of a ligand to a protein or protein complex.

In another embodiment the computer-implemented method can be used to identify ligand binding pockets in a protein structure.

In yet another embodiment the computer-implemented method can be used to calculate the effect of mutations in a protein or protein structure on the stability of a protein or protein structure.

In yet another embodiment the method can be used to refine crystallographic coordinates obtained by Cryo-Electron Microscopy tools.

In yet another embodiment the invention provides a computer-readable storage medium which stores computer-executable instruction that, when executed by at least one processor, causes the processor to perform the herein described methods.

In artificial neural networks an “end to end” architecture is an architecture which defines an unique path of differentiable operations linking the inputs and the outputs

“Pytorch” is an open-source machine learning framework based on the Torch library, used for applications such as computer vision and natural language processing, primarily developed by Meta AI.

“Automatic differentiation”: in mathematics and computer algebra, automatic differentiation (AD), also called algorithmic differentiation, computational differentiation, auto-differentiation or simply autodiff, is a set of techniques to evaluate the derivative of a function specified by a computer program. AD exploits the fact that every computer program, no matter how complicated, executes a sequence of elementary arithmetic operations (addition, subtraction, multiplication, division, etc.) and elementary functions (exp, log, sin, cos, etc.). By applying the chain rule repeatedly to these operations, derivatives of arbitrary order can be computed automatically, accurately to working precision, and using at most a small constant factor more arithmetic operations than the original program. Automatic differentiation is distinct from symbolic differentiation and numerical differentiation. Symbolic differentiation faces the difficulty of converting a computer program into a single mathematical expression and can lead to inefficient code. Numerical differentiation (the method of finite differences) can introduce round-of-errors in the discretization process and cancellation. Both of these classical methods have problems with calculating higher derivatives, where complexity and errors increase. Finally, both of these classical methods are slow at computing partial derivatives of a function with respect to many inputs, as is needed for gradient-based optimization algorithms. Automatic differentiation solves these problems.

“Tensors”: in mathematics, a tensor is an algebraic object that describes a multilinear relationship between sets of algebraic objects related to a vector space. Objects that tensors may map between include vectors and scalars, and even other tensors. There are many types of tensors, including scalars and vectors (which are the simplest tensors), dual vectors, multilinear maps between vector spaces, and even some operations such as the dot product. Tensors are defined independent of any basis, although they are often referred to by their components in a basis related to a particular coordinate system.

In yet another embodiment the invention provides an apparatus comprising control circuitry configured to perform the herein described methods.

Systems of the disclosure can include an intranet-based computer system that is capable of communicating with various software. A computer system includes any type of computing device or communication device. Examples of such a system can include, but are not limited to, super computers, a processor array, distributed parallel system, a desktop computer with LAN, WAN, Internet or intranet access, a laptop computer with LAN, WAN, Internet or intranet access, a smart phone, a server, a server farm, an android device (or equivalent), a tablet, smartphones, and a personal digital assistant (PDA). Further, as discussed above, such a system can have corresponding software (e.g., user software, sensor device software). The software of one system can be a part of, or operate separately but in conjunction with, the software of another system.

Embodiments of the disclosure include a storage repository. The storage repository can be a persistent storage device (or set of devices) that stores software and data. Examples of a storage repository can include, but are not limited to, a hard drive, flash memory, some other form of solid-state data storage, or any suitable combination thereof. The storage repository can be located on multiple physical machines, each storing all or a portion of the database, AI platform, protocols, algorithms, or other stored data according to some example embodiments. Each storage unit or device can be physically located in the same or in a different geographic location. In embodiments, the storage repository may be stored locally, or on cloud-based serveries such as Amazon Web Services.

In one or more example embodiments, the storage repository stores one or more databases, AI Platforms, protocols, algorithms, and stored data. The protocols can include any of a number of communication protocols that are used to send, receive, or send and receive data between the processor, datastore, memory and the user. A protocol can be used for wired and/or wireless communication. Examples of a protocols can include, but are not limited to, Modbus, profibus, Ethernet, and fiberoptic.

Systems of the disclosure can include a hardware processor. The processor of the computer executes software, algorithms, and firmware in accordance with one or more example embodiments. The processor can be a central processing unit, a multi-core processing chip, SoC, a multi-chip module including multiple multi-core processing chips, or other hardware processor in one or more example embodiments. The processor is known by other names, including but not limited to a computer processor, a microprocessor, and a multi-core processor. The processor can also be an array of processors.

In one or more example embodiments, the processor executes software instructions stored in memory. Such software instructions can include generating machine learning models, executing machine learning models, performing analysis on data received from the database, and so forth. The memory includes one or more cache memories, main memory, or any other suitable type of memory. The memory can include volatile or non-volatile memory.

The processing system can be in communication with a computerized data storage system which can be stored in the storage repository. The data storage system can include a non-relational or relational data store, such as a MySQL or other relational database. Other physical and logical database types could be used. The data store may be a database server, such as Microsoft SQL Server, Oracle, IBM DB2, SQLITE, or any other database software, relational or otherwise. The data store may store the information identifying syntactical tags and any information required to operate on syntactical tags. In some embodiments, the processing system may use object-oriented programming and may store data in objects. In these embodiments, the processing system may use an object-relational mapper (ORM) to store the data objects in a relational database. The systems and methods described herein can be implemented using any number of physical data models. In one example embodiment, an RDBMS can be used. In those embodiments, tables in the RDBMS can include columns that represent coordinates. The tables can have pre-defined relationships between them. The tables can also have adjuncts associated with the coordinates.

In embodiments, the systems of the disclosure can include one or more I/O (input/output) devices allow a user to enter commands and information into the system, and also allow information to be presented to the user or other components or devices. Examples of input devices include, but are not limited to, a keyboard, a cursor control device (such as a mouse), a microphone, a touchscreen, and a scanner. Examples of output devices include, but are not limited to, a display device (e.g., a display, a monitor, or projector), speakers, outputs to a lighting network (such as a DMX card), a printer, and a network card. For example, the input devices can be used to enter data on native proteins and mutation sequences and assays. The input devices can also enter wanted functional data for a protein. The output devices can be used to output analysis data and/or engineered protein sequences resulting from AI protein design.

Various techniques are described herein in the general context of software.

Generally, software includes routines, programs, objects, components, data structures, and so forth that perform particular tasks or implement particular abstract data types. An implementation of these modules and techniques can be stored on or transmitted across some form of computer readable media. Computer readable media is any available non-transitory medium or non-transitory media that is accessible by a computing device. By way of example, and not limitation, computer readable media includes computer storage media.

In embodiments, the AI Platform comprises a machine learning method, such as a neural network for effective protein function prediction. In some embodiments, the AI platform includes neural networks, genetic algorithms, decision trees, fuzzy logic, symbolic rules, gradient boosting, support vector machines, and other machine learning based systems. Pluralities and/or combinations of the above may also be used. In embodiments, the AI Platform can use ML frameworks such as, Keras, Caffe, Pytorch, TensorFlow, the Microsoft Cognitive Toolkit, MXNet, Chainer, and Theano, with a Python implementation as the predominant data science language. In embodiments, the AI platform will allow for agnostic integration with other algorithms (such as gradient boosting, SVM, Gaussian processes) and their respective frameworks (XGBoost, SciKit Learn, GPy etc.) by separating data preparation from model creation and by using a NumPy data format common to all of these frameworks. In some embodiments, data preparation tools can be released as a Python package.

Embodiments of the disclosure use protein feature encodings to add physical or biological knowledge to amino acid sequences to create representations amenable to machine learning. As the choice of encoding varies based on the size and diversity of the input, as well as the task, several encoding methods can be implemented, allowing users to test and select the encodings most relevant to their problem. The AI Platform can include the following encodings, for example: one-hot, autoencoders, amino acid property encoders, learned BLOSUM/MSA evolutionary encodings, sequence mutation representation relative to WT, secondary structure/solvent accessible surface area encodings, learned AA embeddings, POOL, Phoenix, and/or structural/graph/topological encodings.

The above-described embodiments of the present invention can be implemented in any of numerous ways. For example, the embodiments may be implemented using hardware, software or a combination thereof. When implemented in software, the software code can be executed on any suitable processor or collection of processors, whether provided in a single computer or distributed among multiple computers. It should be appreciated that any component or collection of components that perform the functions described above can be generically considered as one or more controllers that control the above-discussed functions. The one or more controllers can be implemented in numerous ways, such as with dedicated hardware, or with general purpose hardware (e.g., one or more processors) that is programmed using microcode or software to perform the functions recited above.

One or more processors may be interconnected by one or more networks in any suitable form, including as a local area network or a wide area network, such as an enterprise network or the Internet. Such networks may be based on any suitable technology and may operate according to any suitable protocol and may include wireless networks, wired networks, or fiber optic networks.

One or more algorithms for controlling methods or processes provided herein may be embodied as a readable storage medium (or multiple readable media) (e.g., a computer memory, one or more floppy discs, compact discs (CD), optical discs, digital video disks (DVD), magnetic tapes, flash memories, circuit configurations in Field Programmable Gate Arrays or other semiconductor devices, or other tangible storage medium) encoded with one or more programs that, when executed on one or more computers or other processors, perform methods that implement the various methods or processes described herein.

In some embodiments, a computer readable storage medium may retain information for a sufficient time to provide computer-executable instructions in a non-transitory form. Such a computer readable storage medium or media can be transportable, such that the program or programs stored thereon can be loaded onto one or more different computers or other processors to implement various aspects of the methods or processes described herein. As used herein, the term “computer-readable storage medium” encompasses only a computer-readable medium that can be considered to be a manufacture (e.g., article of manufacture) or a machine. Alternatively, or additionally, methods or processes described herein may be embodied as a computer readable medium other than a computer-readable storage medium, such as a propagating signal.

The terms “program” or “software” are used herein in a generic sense to refer to any type of code or set of executable instructions that can be employed to program a computer or other processor to implement various aspects of the methods or processes described herein. Additionally, it should be appreciated that according to one aspect of this embodiment, one or more programs that when executed perform a method or process described herein need not reside on a single computer or processor but may be distributed in a modular fashion amongst several different computers or processors to implement various procedures or operations.

Executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in various embodiments.

Also, data structures may be stored in computer-readable media in any suitable form. Non-limiting examples of data storage include structured, unstructured, localized, distributed, short-term and/or long term storage. Non-limiting examples of protocols that can be used for communicating data include proprietary and/or industry standard protocols (e.g., HTTP, HTML, XML, JSON, SQL, web services, text, spreadsheets, etc., or any combination thereof). For simplicity of illustration, data structures may be shown to have fields that are related through location in the data structure. Such relationships may likewise be achieved by assigning storage for the fields with locations in a computer-readable medium that conveys relationship between the fields. However, any suitable mechanism may be used to establish a relationship between information in fields of a data structure, including using pointers, tags, or other mechanisms that establish relationship between data elements.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of and “consisting essentially of shall be closed or semi-closed transitional phrases, respectively, as set forth in the United States Patent Office Manual of Patent Examining Procedures, Section 2111.03.

### Examples

### 1. Overview of the Computer-Implemented Algorithm MadraX

### MadraX Groups Energies in 11 Types:

(1) Disulfide bonds, (2) hydrogen bonds, (3) electrostatic interactions, (4) polar solvation, (5) hydrophobic solvation, (6) atomic clashes, (7) backbone entropy, (8) side chain entropy, (9) side chain conformation violation penalty, (10) peptide bond penalty and (11) other Van der Waals interactions with water. An in-depth description of the way how these energies are calculated is described below in the Materials and Methods section.

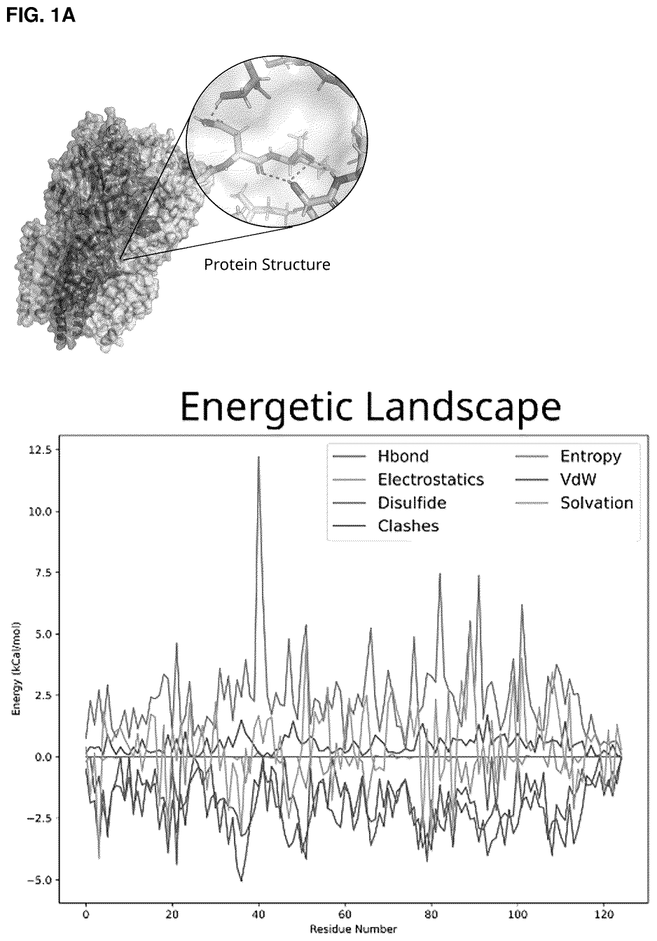

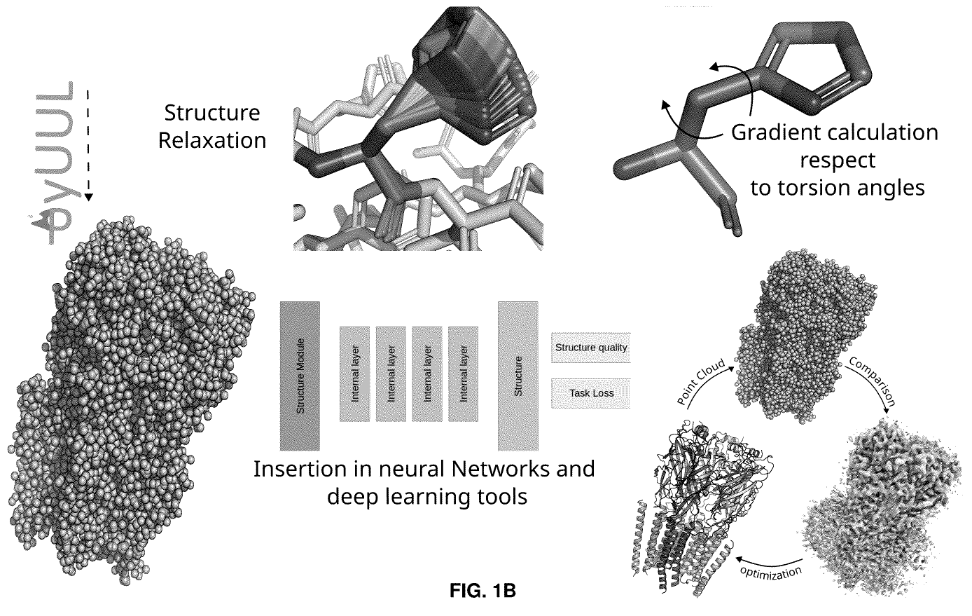

FIGS. 1A and 1B shows an overview of the possible applications of MadraX in structural biology.

### 2. Application of MadraX in Protein Complexes Analysis Obtained by Cryogenic Electron Microscopy

In a first application, we analyzed a task related to cryogenic electron microscopy (cryo-EM), a technique that is growing in popularity in the last years due to its capability of analyzing large protein complexes in their native states. The experimentally obtained electron-density represents the volume occupied by the atoms. However, their actual position has to be inferred taking both the experimental data and the physical constraints into consideration. In other words, researchers typically minimize the total energy of the protein structure or complex, to make the model thermodynamically stable, and maximize the match between the resulting total atomic volume and the electron-density.

In our previous work [6] we proposed a method, called PyUUL, to transform protein structures in tensor-based volumetric representations. We have used this tool here to generate the volume occupied by the atoms of a cryo-EM protein model in a differentiable way. We then built a simple neural network (NN) which rotates such atoms around their torsion angles (phi, psi, omega and chi angles). The network is trained so that the resulting protein conformation, after the application of the rotations, would have a volumetric occupancy, calculated with PyUUL, compatible with the experimental electron-density, while still respecting the biophysical constraints of protein structures. The two volumes are compared using Chamfer distance loss implemented in PyTorch3D [7], a loss function often used to train NNs in computer vision, while biophysics is evaluated by MadraX. The final result is a method that iteratively refines the position of the atoms of a cryo-EM model in accordance with the electron-density in an automatic way and without any manual intervention. A more elaborate discussion about this application of MadraX is available in example 3 and details about the NN are outlined in the materials and methods section.

Yet another example of usage of MadraX coupled with a NN, is the identification of amino acid residues in a protein fold which are involved in ligand binding, this is explained in example 6.

### 3. Application of MadraX in Cryo-EM Model Refinement

In this example the use of MadraX is illustrated inside a neural network. To our knowledge, this is the first fully differentiable forcefield that allows automatic differentiation (unlike a forcefield like FoldX). The fact that MadraX is built as a PyTorch module allows us to use it as a loss function for an optimization and to couple it with methods that are typical for computer vision, such as cloud point analysis. This approach shows that it is possible to use machine learning to automate and standardize the process with which atom positions are assigned in cryo-EM, taking the raw experimental data into account. During the relaxation of the model, the Chamfer distance gets reduced by 0.128. This means that the correspondence between the atomic volume and the electron density is increased. At the same time, Madrax energy gets reduced by 25 kcal/mol, showing that optimization is also beneficial for the stability of the conformation.

### 4. Validation of Protein Stability Optimization Using MadraX

MadraX is implemented as a PyTorch module and therefore it maintains the properties of auto-differentiation, typical of NN layers. These properties allow the application of general algorithms, such as the optimizers developed by Facebook or Google, to structural biology problems. Additionally, we can automatically calculate the gradient of the energy with respect to any arbitrary variable. This means that, given a protein P, described by atom coordinates X, MadraX can take these coordinates as input and provide an energy E. If we calculate the gradient SE/8X, we define how E changes with respect to the position of the atoms. If we use one of the aforementioned generic optimizer provided by PyTorch (i.e. Adam [8]) to minimize E by moving the coordinates X, we obtain an extremely fast gradient-based structure optimizer. Alternatively, we can also differentiate E with respect to the torsion angles, thereby maintaining the geometry of covalent bonds within the protein. Both these optimization procedures are purely gradient-based and do not require any fragments database as support. The materials and methods section provides more details about the implementation of this procedure.

In order to confirm that MadraX maintains the properties and predictive capabilities of a standard forcefield, we estimated its capability of predicting the effect of mutations on the stability of proteins. For this purpose, we downloaded a dataset of 342 mutations available on Protherm and downloaded from [20], along with the respective wild type structures. We implemented the mutations in the respective wild type structures using MadraX utility function mutationEngine.mutate. We then defined a NxCxRxMx5 tensor, where N is the number of protein structures, C the maximum number of chains, R the maximum number of residues and M the maximum number of mutations per protein. This tensor represents the rotation applied to the Chi angles of every residue. We used MadraX utility function mutationEngine.rotator to implement the rotation. This function is implemented so that all amino acids, but the mutated ones, are shared between the mutant and the wild type and therefore rotated in the same way. This gives a huge boost to computational speed and memory usage, along with the fact that it reduces the variability in AG estimation, caused by stochastic optimizers. We then used a PyTorch optimizer (Adam) with a learning rate of 10-2 for 50 epochs to find the set of rotations that minimize the energy of both mutant and wild type structures. This procedure makes sure that both wild type and mutant structures are in a conformation associated with an energetic minimum. In order to estimate the effect of the mutation we simply took the energy difference between the mutant and the wild type.

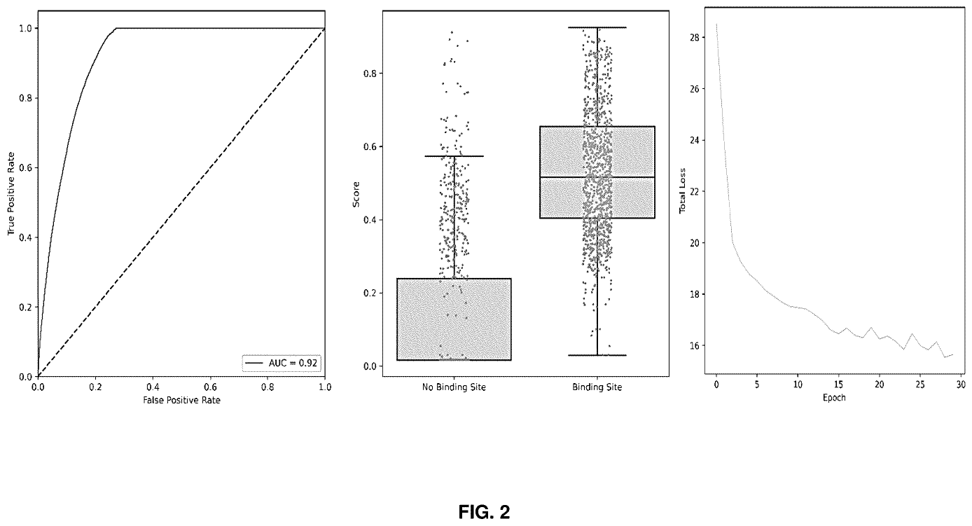

The correlation between the predicted ΔΔG and the experimental one is 0.51. The performances are in line with the ones of the best forcefield-based stability predictors. FIG. 2 shows the scatter plot between the predicted and experimental ΔΔG. Despite its high performances, this is just an example of the usefulness of the automatic differentiation function of MadraX and it should not be interpreted as a predictive tool. However, it is indeed technically possible to build a fast- and easy-to-use stability predictor with MadraX. Specifically, we believe such a tool would largely benefit from an ad hoc policy to choose which residues have to be optimized and which ones are likely to have a different conformation in the mutant. Such a policy, currently absent in this example, would reduce the number of dimensions the optimizer has to work in, along with the number of differentiation operations that would be required. In other terms, a full protein optimization, rotating the side chain of every single residue, is never done in protein stability predictors, such as Rosetta or FoldX. This is due to the fact that the large amount of rotations involved would negatively affect the capability of the optimizer to find the absolute minimum of the energy function.

For this reason, limiting the amount of rotations the optimizer has to deal with would improve both the speed and the performances of the predictor.

### 5. Use of MadraX to Calculate Molecular Motions

Up until now, we have been using the automatic gradient calculation only in the context of gradient descent optimizers. Derivatives, however, are also used to calculate physical quantities. In particular, the speed is defined as the derivative of space with respect to time (δ5/δt), or, in more practical words, the distance travelled by an object in a small amount of time. In a physical molecular simulation, commonly referred to as molecular dynamics, atoms are assigned with an initial speed and the algorithm simulates the evolution of the system, taking the interaction forces between atoms into account.

When calculating the gradient of MadraX's energy with respect to the atom position, we obtain a pseudo speed, given by the attractive and repulsive forces between atoms, since it tells us how much every atom moves in a single time step (an epoch). Similarly, if we calculate the gradient of MadraX's energy with respect to the torsion angles, we obtain a pseudo angular speed. To do so, we first needed to tune initial speed in accordance with the energy available in the system. We therefore tried to simulate pseudo motions of atoms by adding an initial angular speed to each torsion angle in the protein and letting the system evolve by itself. Such initial speed is correlated to the kinetic energy available in the system. We therefore defined a coefficient that we call ‘temperature coefficient’ which describes the magnitude, in average, of the initial motion of atoms. Subsequently, we calculated the gradient of MadraX's energy with respect to the torsion angles of the protein. This value defines where and how each atom is “pushed” by the molecular forces and therefore it represents the angular speed given by atomic interaction.

We can sum the two speeds acting on each torsion angle to find the resulting angular speed. The position of each atom can be updated as in the previous example making use of a PyTorch optimizer (In this case we used SGD).

We can repeat the same operations, using the new angular speed as initial speed and obtain pseudo-trajectories that evolve in time.

As for protein relaxation, this motion simulation does not require any rotamer database: the motion is continuous in space, it is purely gradient-based and it is possible thanks to the differentiability of MadraX.

### 6. Use of MadraX to Predict Amino Acid Residues Involved in Ligand Binding

A typical class of problems in bioinformatics consists of the prediction of residue-based emergent properties. This is the case for several tasks, such as prediction of disorder [11, 12], protein aggregation [13, 14] or protein-protein interaction patches [15]. ML methods applied to these tasks often do not use structural data. The few exceptions [16, 17] simply extract features such as secondary structure or solvent accessibility from the protein structures. This simple feature extraction does not reflect the thermodynamic complexity of a protein, moreover the choice of these approaches is often related to the lack of proper supporting tools to transform the complex structural data into sequential data. The real complexity of the protein fold, including the forces that stabilize it and the actual interactions between amino acids, is therefore often neglected. In order to give a simple and straightforward introduction to MadraX, we built a MadraX-based NN to address a sequence-oriented task, specifically predicting the residues involved in ligand binding.

The network has been trained on the data downloadable from https://zhanggroup. org/COACH/benchmark/. The training set consists of 400 proteins, of which two got removed for issues with the annotation, while the testing set contains 500 proteins. Every residue is annotated with a 1, if it is involved in the binding, or with a 0, if otherwise. The problem consists of training a NN on the training set and predicting which residues, belonging to the proteins of the test set, are involved in binding.

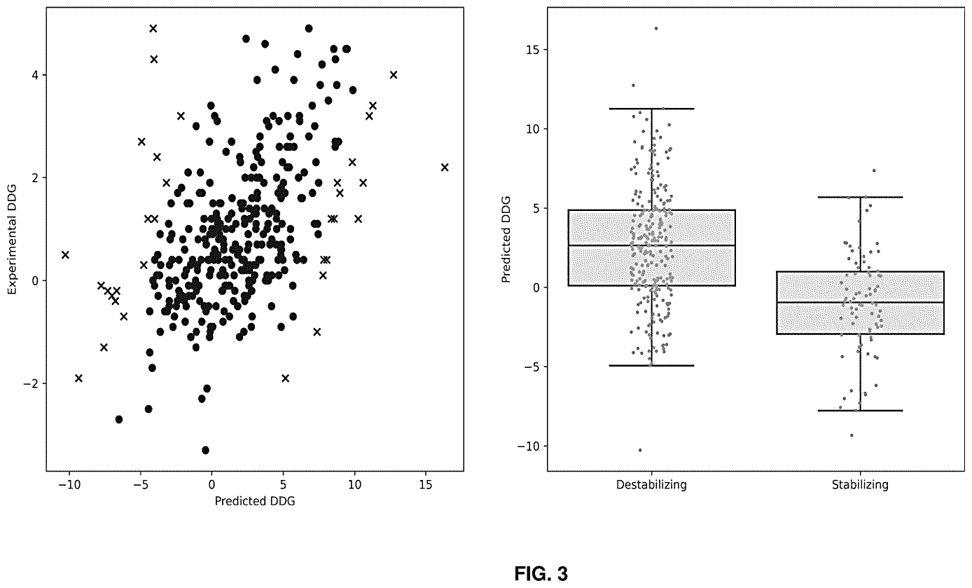

For this purpose, we calculated the energetic profile for every protein in the dataset using MadraX, which provides a per-residue estimation of the energies involved as a tensorial representation. We use this tensorial representation, along with the protein sequence, as input for a 3 layers 1-D convolutional NN with hyperbolic tangent activation, dropout of 0.01 and kernel of 5. The output of this network is taken as input by a 4 layers feed-forward module with 30 hidden neurons, each with hyperbolic tangent activation, dropout of 0.01 and layer normalization. The network ends in a single neuron, assigning the final probability of interacting with the ligand to each residue. The network has been trained for 200 epochs with Adam optimizer and learning rate of 10-4, using a weighted Binary cross entropy with logits, with weights 1 and 15 for the negative and positive class respectively to take into account the class unbalance as loss function. The final AUC of the validation is 0.92. In accordance to the performances of the state of the art predictors reported in the COACH paper [18], this network would rank as first, with the COACH algorithm having an AUC of 0.87. FIG. 3 shows the ROC curve and the box-plot of the scores assigned to positively and negatively annotated residues.

Given the fact that the datasets are highly unbalanced through the negative class (there are about 15 negatively annotated residues for every positive one), predictors developed in this subject should be considered as prioritization tools instead of classical binary classifiers. The goal is therefore to give on average a higher score to residues that are involved in binding, so that the user can focus on a restricted number of residues for experimental analysis. This is the reason why we decided to use a rank-based estimator, such as the AUC, to evaluate the performances.

### Methods

### Definition of the Energies

MadraX provides an estimation of the energies that stabilize a biological molecule or complex. The energies are defined by hard coding the biophysical formulas in tensorial form, therefore no machine learning is involved. MadraX groups energies in 11 classes, six of which have been taken from the FoldX force-field and reimplemented in an efficient way in tensorial form. For a detailed description of the Hydrogen Bonds, Electrostatic Interactions, Disulfide Bonds, Van der Waals Energies, Side Chain Entropy and Polar and Hydrophobic Solvation we refer to the FoldX article [4].

In the next paragraphs we will describe how the remaining energies are modelled and calculated.

### Clashes

Clashes occur when two atoms are too close and they partially overlap their Van der Waals spaces. Clashes should almost never be present in high quality protein models and atom overpacking is expected to give a huge penalty on protein stability. MadraX defines the presence of clashes and their magnitude in accordance with the minimum distance between specific pairs of atoms observed in high resolution crystal structures, in the light of their atomic radius.

To do so, we first downloaded 5000 high resolution (<1.5 A) x-ray protein structures from PDB and, for every atom pair within 5 A from each other, we collected the difference between their distance and the sum of their radius.

We removed from the calculations all the pairs that were connected by a covalent bond (with the exclusion of disulfide bonds) or part of a rigid geometrical structure, such as to atoms belonging to the same aromatic ring or the carbonyl and carbon delta of prolines.

We also excluded from the calculation pair of atoms that were part of the backbone of the same residue.

We then grouped them in six different group: 1) pairs belonging to the same residue, 2) atoms forming an hydrogen bond, 3) atoms forming a disulfide bond, 4) atoms of the backbone of consecutive residues, 5) donor-acceptor pairs not forming an hydrogen bond and 6) all the rest. For each group, we calculated the 10th percentile of the distances-sum-of-radii difference, which we call correction. When the clash energy between two atoms is evaluated, we assign an exponentially growing energetic penalty to all the pairs associated with a distance that is lower than the value of its group, using the following formula:

$$w {e}^{10 * ( {R}_{i} + {R}_{j} - {d}_{i , j} - {t}_{g} )}$$

where w is a scaling factor, Rᵢ and Rⱼ are the radius of atoms i and j respectively, dᵢⱼ is the distance between atom i and atom j and t_{g} is the correction for the group to which the atom pair i, j belongs.

### Backbone Entropy

We calculate the backbone entropy as the log-probability of observing a residue having specific backbone torsion angles. The distribution of angles is estimated from the high resolution crystal structures already used for the estimation clashes and we used a pytorch-differentiable kernel density estimation (KDE) method based on masked autoregressive flow [9]. For every residue type, we define two distributions: one (with 2 dimensions) for the Phi/Psi angles and one for the Omega angle. The latter results in two very narrow Gaussian distributions around-180 and 180 degrees. The final backbone entropy is the sum of these two values. In more mathematical terms,

$${Entropy}_{i} = - {w}_{1} ln ( {KDE}_{Ω} ) - {w}_{2} ln ( {KDE}_{Φ Ψ} )$$

wherein w1 and w2 are two scaling factors, omegaKDE and phiPsiKDE are the two aforementioned probability distributions and phi, psi and omega are the backbone torsion angles.

### Peptide Bond Violation

Peptide bonds are covalent interactions between the amino and carbonyl groups of two consecutive residues and their geometry is strongly constrained by atom hybridization and optimal binding distances. These values, however, can have small variations around the optimal values without disrupting protein stability. In order to allow such variability, we give a penalty to stability based on the deviation from the optimal geometrical values. Specifically, we consider two angles (CA-N-Cₚ and C_{Ap}-Cₚ-N, where p indicates an atom of the previous residue) and the distance between the nitrogen and the carbon involved in the peptide bond. For each of these values, we built a Gaussian distribution (Gi), based on the values observed in the dataset of aforementioned high resolution crystal structures. The final penalty Vis then defined as

$$V = wLn ( {G}_{i} ( {X}_{i} ) )$$

Wherein w is a scaling factor, i is the estimator under examination (one of the two angles or the bond length), G; is the Gaussian distribution for the estimator i and Xᵢ is the value of the estimator i for that specific peptide bond.

### Side Chain Conformation Violation Penalty

We define the side-chain conformation of a residue by its Chi torsion angles. Amino acid side-chains have up to 5 Chi torsion angles for arginine. This means that, assuming a fixed covalent bond geometry and length in the side chain, every possible conformation can be defined in a space with up to 5 dimensions, each of which represents a torsion angle. In reality, not all the conformations are physically possible and a large part of them are never or very rarely found in proteins. In order to account for this effect we introduced a side-chain conformation penalty that is calculated using the same KDE approach described in backbone entropy, but this time the axis represents the Chi angles of the amino acid under scrutiny. The estimation of the probability distribution has been performed using the same dataset of high resolution crystal structures described in the previous sections. The final penalty for a specific residue is again given by the negative logarithmic probability provided by the kernel. In mathematical terms,

$${SCviolatrion}_{i} ( X ) = - {w}_{i} ln ( {KDE}_{i} ( X ) )$$

Wherein w_i is a scaling factor for residue i, KDE; is the aforementioned probability distribution, chi are the torsion angles of the side chain.
   Cryo-EM Model Refinement with End-to-End Deep Learning

We downloaded the protein complex model and the map file representing the electron-density from the PDB web site (PDB-ID: 6KNF). We transformed the map file into a Numpy array using the Emda Python package. In this application of MadraX, we have a NN interacting in an end-to-end fashion with the forcefield module. The network is in charge of generating the set of rotations that minimize the loss functions and its weights are iteratively refined in order to reach this goal. The network consists of a feed-forward neural network (FFNN) with 3 layers and Rectified Linear Unit (ReLU) activation. Since we are optimizing a single protein and the network is only in charge of “proposing” sets of rotations that minimize the loss functions, it does not take any input. The FFNN therefore starts from a single neuron that is always active (always a value of 1). The FFNN ends in a layer with a number of neurons equal to the number of residues in the proteins times 8 and it defines the set of rotations to perform on each torsion angle of each residue.

Different from the previous example, in this case also the backbone is rotated and we use the MadraX utility function rotator to rotate the starting complex conformation. The newly generated structure is then converted into a point cloud using PyUUL [6]. The structure is then evaluated by two loss function:
(1) MadraX itself, that provides the energy of the complex, (2) the Chamfer distance between the PyUUL's cloud point and the experimental electron-density of the protein complex, turned into a cloud point taking the center of the occupied voxels as points. Occupied voxels have been defined applying the threshold suggested by the authors of the cryo-EM experiment in the validation report, available on the relative PDB web page.
   Protein Relaxation with MadraX

For protein relaxation we started downloading the protein structure file from the Protein Data Bank. The file has been parsed using MadraX utility function utils.parsePDB, which provides atom coordinates in tensorial form and a list of atom names. We used the atom names to pre-calculate atom information and hashing using the function dataStructures.createinfotensors. We then created a tensor of shape NxCxRx1x5, where N is the number of protein structures (1 in this case), C the number of chains and R the number of residues of the target protein. This tensor represents the rotation applied to the Chi angles of every residue.

We estimated the energy of the protein using MadraX main object Forcefield and we calculated the gradient of such energy with respect to the aforementioned tensor. We use pytorch optimizer object torch.optim.Adam to perform a gradient-descent-based optimization and find the set of rotations that minimize the energy of the protein. We used the MadraX utility function mutationEngine.rotator to implement such rotation. The optimization has been run for 100 epochs. This procedure is provided as a tutorial in the online documentation of MadraX.

Motion Simulation with MadraX

In order to simulate motion of the atoms using a pytorch setup, we started from the structure of human SH3 which we downloaded from the PDB. The positions of the atoms have been parsed using MadraX's utility function $utils.parsePDBS and we precalculated the information required for the energy calculation with the $dataStructures.createinfotensors$ function. We then relaxed the protein structure as described in the previous section for 500 epochs with a learning rate of 0.001, reduced during the optimization using the $torch.optim.Ir_scheduler.ReduceLROnPlateau$ pytorch scheduler with patience of 100 and factor of 0.5. This procedure has the goal of obtaining a conformation as close as possible to a steady state, in which the total energy has a gradient close to 0 respect to all the torsion angles of the protein. Pytorch, however, uses stochastic numerical optimizers and this procedure does not completely obliterate the gradient. This means that in our motion simulation, some atoms would start with a higher initial potential energy. To compensate for this, we added a starting speed (gradient) to every torsion angle, giving to each one of them the same energy of the torsion angles with the maximum gradient. Now that the system is homogeneous from the energetical point of view, we add a gaussian perturbation to the gradient of every torsion angle. For each torsion angle, the perturbation is sampled from a normal distribution and then multiplied by a scalar that we call temperature coefficient. The latter defines the magnitude of the perturbation. We then let the simulation iteratively evolve, and at every iteration we calculate the gradient of the energy calculated by MadraX with respect to a rotation of the torsion angles. This represents the motion given by the forces of interaction between the atoms. We add this value to the gradient of the previous iteration, that represents the motion at the beginning of the iteration step. We let the system evolve in this way for 500 epochs. The actual motion is implemented using SGD optimizer from pytorch, with learning rate of 10\hat{ }−7 and 10\hat{ }−5 for backbone and side chain torsion angles respectively.

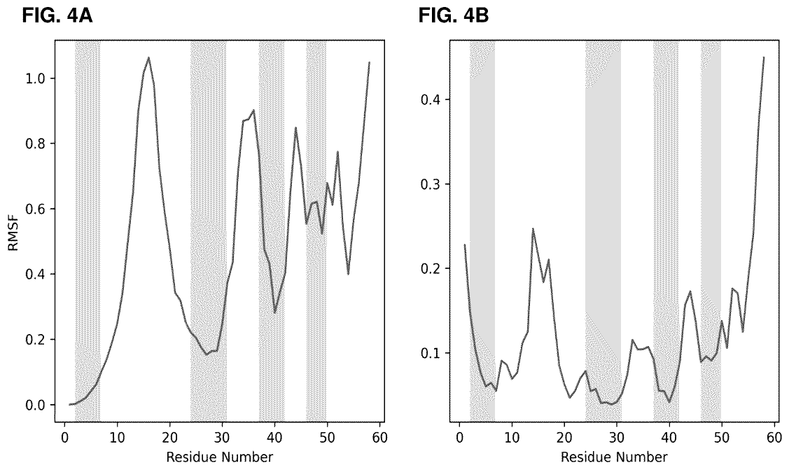

Discussion: SH3 domain is a relatively small protein and it has been largely investigated from the dynamics point of view. One of these analyses can be found in In this article, authors show the results of MD simulations performed with Gromax [22]. Specifically, they provide the root mean square fluctuation (RMSF) of each residue, which estimates how much every residue moves during a simulation. We performed the same analysis with the pseudo-motions calculated by MadraX, and the results are reported in FIG. 4. In our simulation, we assign pseudo angular speed to torsion angles. This means that the first residue is the center of rotation and therefore it does not move by definition. FIG. 4A shows the RMSF of the residues generated with MadraX, while FIG. 4B shows the RMSF obtained performing a structural alignment of the generated intermediate conformations using Pymol's “fit” function [23]. It is important to note that the structural alignment, while it provides a more realistic estimation of the flexibility of the protein termini, introduces alignment errors and it therefore adds noise. The plots shown in FIG. 4 are extremely similar to the ones shown in FIG. 3 of [21], demonstrating that there is concordance between our pseudo-motions and actual molecular dynamics simulation trajectories performed with MD tools.

### REFERENCES

1. Jumper, J. et al. Highly accurate protein structure prediction with alphafold. Nature 596, 583-589 (2021).
2. Orlando, G., Raimondi, D. & Vranken, W. Observation selection bias in contact prediction and its implications for structural bioinformatics. Scientific Reports 6 (2016).
3. Rohl, C. A., Strauss, C. E., Misura, K. M. & Baker, D. Protein structure prediction using rosetta. Methods in enzymology 383, 66-93 (2004).
4. Guerois, R., Nielsen, J. E. & Serrano, L. Predicting changes in the stability of proteins and protein complexes: a study of more than 1000 mutations. Journal of molecular biology 320, 369-387 (2002).
5. Paszke, A. et al. Automatic differentiation in pytorch (2017).
6. Orlando, G. et al. Pyuul provides an interface between biological structures and deep learning algorithms. Nature communications 13, 1-9 (2022).
7. Ravi, N. et al. Accelerating 3d deep learning with pytorch3d. arXiv preprint arXiv: 2007.08501 (2020).
8. Kingma, D. P. & Ba, J. Adam: A method for stochastic optimization. arXiv preprint arXiv: 1412.6980 (2014).
9. Papamakarios, G., Pavlakou, T. & Murray, I. Masked autoregressive flow for density estimation (2017). URL https://arxiv.org/abs/1705.07057.
10. Warshamanage, R., Yamashita, K. & Murshudov, G. N. Emda: a python package for electron microscopy data analysis. Journal of Structural Biology 214, 107826 (2022).
11. Walsh, I., Martin, A. J., Di Domenico, T. & Tosatto, S. C. Espritz: accurate and fast prediction of protein disorder. Bioinformatics 28, 503-509 (2012).
12. Orlando, G., Raimondi, D., Codice, F., Tabaro, F. & Vranken, W. Prediction of disordered regions in proteins with recurrent neural networks and protein dynamics. Journal of Molecular Biology 434, 167579 (2022).
13. Fernandez-Escamilla, A.-M., Rousseau, F., Schymkowitz, J. & Serrano, L. Prediction of sequence-dependent and mutational effects on the aggregation of peptides and proteins. Nature biotechnology 22, 1302-1306 (2004).
14. Orlando, G., Silva, A., Macedo-Ribeiro, S., Raimondi, D. & Vranken, W. Accu-rate prediction of protein beta-aggregation with generalized statistical potentials. Bioinformatics 36, 2076-2081 (2020).
15. Hashemifar, S., Neyshabur, B., Khan, A. A. & Xu, J. Predicting protein-protein interactions through sequence-based deep learning. Bioinformatics 34, 1802-1810 (2018).
16. Dehouck, Y., Kwasigroch, J. M., Gilis, D. & Rooman, M. Popmusic 2.1: a web server for the estimation of protein stability changes upon mutation and sequence optimality. BMC bioinformatics 12, 1-12 (2011).
17. Zambrano, R. et al. Aggrescan3d (a3d): server for prediction of aggregation prop-erties of protein structures. Nucleic acids research 43, W306-W313 (2015).
18. Yang, J., Roy, A. & Zhang, Y. Protein-ligand binding site recognition using comple-mentary binding-specific substructure comparison and sequence profile alignment. Bioinformatics 29, 2588-2595 (2013).
19. Gromiha, M. M. et al. Protherm: thermodynamic database for proteins and mutants. Nucleic acids research 27, 286-288 (1999).
20. Pucci, F., Bernaerts, K. V., Kwasigroch, J. M. & Rooman, M. Quantification of biases in predictions of protein stability changes upon mutations. Bioinformatics 34, 3659-3665 (2018).
21. Chu, W. T., Zhang, J. L., Zheng, Q. C., Chen, L., & Zhang, H. X. (2013). Insights into the folding and unfolding processes of wild-type and mutated SH3 domain by molecular dynamics and replica exchange molecular dynamics simulations. PLOS One, 8 (5), e64886.
22. Abraham, Mark James, et al. “GROMACS: High performance molecular simulations through multi-level parallelism from laptops to supercomputers.” SoftwareX 1 (2015): 19-25.
23. Schrödinger, L., & DeLano, W. (2020). PyMOL. Retrieved from http://www.pymol.org/pymol

1. A computer-implemented method to determine the stability of the folding of a protein or a protein complex, the method comprising:
i) obtaining the crystallographic atom coordinates of a protein structure or the structure of a protein complex,
ii) calculating biophysical energy values of each of the amino acid residue interactions present in said protein or protein complex wherein the parameters comprise calculation of hydrogen bonds, electrostatic interactions, polar solvation, hydrophobic solvation, atomic clashes, disulfide bonds, backbone entropy, side chain entropy and other Van der Waals interactions with water, organized in higher dimensional tensor data structures, and
iii) introducing the tensor data structures into a deep learning algorithm in an end-to-end manner,
iv) calculating the gradient of the biophysical energy values with respect to a variation in the atom coordinates obtained in step a) with the use of an automatic differentiation engine,
v) calculating the stability of the folding of a protein or a protein complex.

2. The method according to claim 1, further comprising for identifying binding pockets in a protein structure

3. The method according to claim 1, further comprising calculating the effect of mutations on the stability of a protein structure or a protein complex.

4. The method according to claim 1, further comprising refining Cryo-EM protein complexes

5. A computer-readable storage medium which stores computer-executable instruction that, when executed by at least one processor, cause the processor to perform the method claim 1.

6. An apparatus comprising control circuitry configured to perform the method of claim 1.

7. The method according to claim 1, further comprising measuring the stability of the folded protein or protein complex.
